(19) United States
(12) Patent Application Publication
(10) Pub. No.: US 2026/0034293 A1
(43) Pub. Date: Feb. 5, 2026

(54) ENDOLUMINAL TREATMENT SYSTEMS, DEVICES, AND RELATED METHODS

(71) Applicant: Boston Scientific Scimed, Inc., Maple Grove, MN (US)

(72) Inventors: Sean P. FLEURY, Nashville, TN (US); Jeff GRAY, Sudbury, MA (US); Ryan V. WALES, Shrewsbury, MA (US)

(73) Assignee: Boston Scientific Scimed, Inc., Maple Grove, MN (US)

(21) Appl. No.: 19/283,380

(22) Filed: Jul. 29, 2025

Related U.S. Application Data
(60) Provisional application No. 63/678,196, filed on Aug. 1, 2024.

(51) Int. Cl. A61M 1/00; A61M 25/10

(52) U.S. Cl. CPC A61M 1/916; A61M 1/915; A61M 25/10

(57) ABSTRACT

A medical device may include a tube defining a lumen. The tube may be configured to couple to a source of suction. The medical device may include a porous body coupled to a distal portion of the tube. The porous body may include a plurality of openings in fluid communication with the lumen. The medical device may further include a wire coupled to the distal portion of the tube. The wire may be configured to move the distal portion of the tube and the porous body via a pulling force applied to the wire. The pulling force applied to the wire may exert a force on the tube that is at a non-zero angle relative to a central longitudinal axis of the tube.

### CROSS-REFERENCE TO RELATED APPLICATION(S)

This application claims the benefit of priority to U.S. Provisional Application No. 63/678,196, filed on Aug. 1, 2024, which is incorporated by reference herein in its entirety.

### TECHNICAL FIELD

Various aspects of this disclosure relate generally to minimally invasive medical devices and related methods of use. In particular, aspects of the disclosure relate to medical devices and methods for endoscopic medical procedures, such as closing a wound or otherwise treating tissue.

### BACKGROUND

Endoscopic and open surgical procedures of the gastrointestinal (GI) tract include, e.g., colonic resection, bariatric surgery, esophagectomy, gastric bypass, and sleeve gastrectomy, among others. These procedures may result in perforation, post-surgical anastomotic leaks, or other wounds of the GI tract. Patients with perforations, post-surgical anastomotic leaks, and/or other wounds in the GI tract have high mortality rates with limited treatment options. Options include endoscopic placement of clips or stents, endoscopic sutures or sealants, or surgical re-operation. Surgery is relatively invasive and has high morbidity and mortality rates. While endoscopic stent placement is a less invasive option, the stent can migrate from the intended location and/or wall off infection at a treatment site, inhibiting drainage.

### SUMMARY

Each of the aspects disclosed herein may include one or more aspects of the features described in connection with any of the other disclosed aspects.

In an aspect, a medical device may include a tube defining a lumen. The tube may be configured to couple to a source of suction. The medical may include a porous body coupled to a distal portion of the tube. The porous body may include a plurality of openings in fluid communication with the lumen. The medical device may further include a wire coupled to the distal portion of the tube. The wire may be configured to move the distal portion of the tube and the porous body via a pulling force applied to the wire. The pulling force applied to the wire may exert a force on the tube that is at a non-zero angle relative to a central longitudinal axis of the tube.

Any of the devices, systems, or methods disclosed herein may include any of the following features, alone or in any combination. The medical device may further include an attachment device extending through at least a portion of the porous body and the tube. The attachment device may be coupled to the wire. The attachment device may be proximate a distalmost end of the porous body. The attachment device may include a stem portion and a head portion. The stem portion may extend through the lumen of the tube approximately perpendicularly the central longitudinal axis of the tube. The head portion may be disposed exterior of the tube and may extend perpendicular to the stem portion. Ends of the wire may be coupled to the attachment device forming a loop. The loop may extend proximally from the attachment device when the porous body is within a body lumen. The lumen of the tube may be a first lumen of the tube and the tube may further define a second lumen. The wire may be received within the second lumen. The tube may include a slit extending through a wall of the tube and in fluid communication with the second lumen, and the wire may be movable from a position within the second lumen to a position outside of the tube via the slit. The wire may be configured to transition the distal portion of the tube between a linear configuration and a bent configuration. The medical device may further include a handle coupled to a proximal end of the tube. The handle may include an actuator and the wire may extend proximally from the distal portion of the tube to the actuator. The actuator may be configured to move the wire in a proximal direction or a distal direction. The handle may include a port in fluid communication with the lumen of the tube and configured to couple to the source of suction. A portion of the wire may extend outside of the tube and within the porous body. The portion of the wire may be configured to cut the porous body.

In another aspect, a medical device may include a porous body having a plurality of openings and a tube defining a lumen in fluid communication with the plurality of openings of the porous body. A proximal end of the tube may be configured to couple to a source of suction and a distal end of the tube may be coupled to the porous body. The medical device may further include a helical body coupled to the distal end of the tube.

Any of the devices, systems, and methods disclosed herein may include any of the following features, alone or in any combination. The helical body may extend distally from a distal opening of the lumen of the tube. The helical body may extend around the distal end of the tube.

In a further aspect, a medical system may include a tube extending from a proximal end to a distal end and defining a lumen. The proximal end of the tube may be configured to couple to a source of suction. The medical system may include a porous body coupled to a distal end of the tube. The porous body may include a lumen at a distal portion of the porous body in fluid communication with the lumen of the tube. The lumen of the porous body may include an opening at a distalmost end of the porous body. The medical system may further include a removal device. The removal device may include a shaft extending from a proximal end to a distal end and defining a lumen. The proximal end of the shaft may be configured to couple to a source of fluid. The removal device may further include an inflatable balloon coupled to the distal end of the shaft and in fluid communication the lumen of the shaft. The inflatable balloon may be configured to extend through the lumen of the tube and the lumen of the porous body.

### BRIEF DESCRIPTION OF THE DRAWINGS

The accompanying drawings, which are incorporated in and constitute a part of this application, illustrate exemplary aspects of this disclosure and together with the description, serve to explain the principles of the present disclosure.

FIG. 1A illustrates an exemplary medical device, in accordance with some aspects of the present disclosure.

FIG. 1B illustrates the medical device of FIG. 1A at a target site, in accordance with some aspects of the present disclosure.

FIG. 2 illustrates a cross-sectional view of an exemplary vacuum tube, in accordance with some aspects of the present disclosure.

FIG. 3 illustrates another exemplary medical device, in accordance with some aspects of the present disclosure.

FIG. 4 illustrates another exemplary vacuum tube, in accordance with some aspects of the present disclosure.

FIG. 5 illustrates an exemplary coupler, in accordance with some aspects of the present disclosure.

FIGS. 6A and 6B illustrate an exemplary medical system, in accordance with some aspects of the present disclosure.

FIGS. 7A and 7B illustrate a further exemplary medical device, in accordance with some aspects of the present disclosure.

FIG. 8 illustrates another exemplary medical system or device, in accordance with some aspects of the present disclosure.

### DETAILED DESCRIPTION

Particular aspects of the present disclosure are described in greater detail below. The terms and definitions provided herein control, if in conflict with terms and/or definitions incorporated by reference. Wherever possible, the same or similar reference numbers will be used through the drawings to refer to the same or like parts.

The terms “proximal” and “distal” are used herein to refer to the relative positions of the components of exemplary medical devices. As used herein, “proximal” refers to a position relatively closer to the exterior of the body or closer to an operator using the medical device. In contrast, “distal” refers to a position relatively further away from the operator using the medical device, or closer to the interior of the body.

As used herein, the terms “comprises,” “comprising,” “including,” “includes,” “having,” “has” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.”

Further, relative terms such as, for example, “about,” “substantially,” “approximately,” etc., are used to indicate a possible variation of ±10% in a stated numeric value or range.

The present disclosure includes medical devices for endoluminal vacuum therapy (EVT or EVAC, and referred to herein as EVAC). EVAC is a procedure to treat wounds, such as post-surgical leaks or perforations in the gastrointestinal tract (GI) following a surgical or endoscopic procedure, such as colonic resection, bariatric surgery, or esophagectomy. For example, the medical devices herein may include a porous body (e.g., a sponge, foam, or other like material) coupled to a distal end of a tube (e.g., a vacuum tube). The porous body may be placed into a wound cavity (e.g., a target site including a perforation, a leak, a cyst, an anastomosis, or other wound). Placement of the porous body may be via a catheter, scope (endoscope, bronchoscope, colonoscope, duodenoscope, gastroscope, etc.), tube, or sheath, inserted into the GI tract via a natural orifice. The orifice may be, e.g., the nose, mouth, or anus, and the placement may be in any portion of the GI tract, including the esophagus, stomach, duodenum, large intestine, or small intestine. Placement of the porous body may also be in other organs reachable via the GI tract (e.g., colon, biliary tract, appendix). The tube may be coupled to a collection container in conjunction with a negative pressure source, and negative pressure may be applied to the wound cavity.

The porous body, along with the negative pressure, may accelerate healing by encouraging local granulation of tissue at the wound cavity. The negative pressure may remove fluid and/or material from the wound cavity, and may pull portions of the wound cavity inwards towards the porous body, reducing the size of the wound cavity. The porous body may be replaced with smaller sizes of porous bodies as the wound heals and closes. Features of the medical devices herein may facilitate easy removal of the porous body from the wound cavity and/or reduce the time needed to remove the porous body from the wound cavity. For example, features of the medical devices herein may help prevent the tube from separating from the porous body, e.g., when the tube is pulled proximally, and/or may assist with peeling or otherwise detaching the porous body from tissue (e.g., granulated tissue) within the wound cavity. Conventional EVAC devices may be susceptible to the tube separating from the porous body when the tube is pulled proximally.

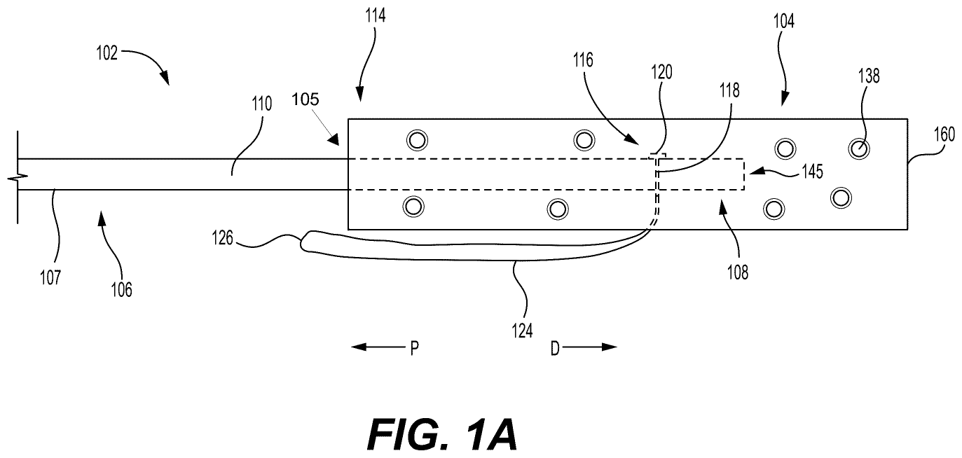

FIG. 1A illustrates a distal portion of an exemplary medical device 102. FIG. 1B illustrates the distal portion of medical device 102 within a body lumen 150 (e.g., the esophagus) to treat a wound cavity 155 (e.g., a leak, an esophageal fistula, etc.). Medical device 102 includes a sponge 104, or other porous body, and a tube 106 or other type of conduit. Sponge 104 may include openings 138 on an outer surface and/or interior thereof. Openings 138 may be any hole, pore, or channel. Openings 138 may include interconnecting channels and/or pores throughout sponge 104. For example, openings 138 may be pores of sponge 104. Openings 138 may have different sizes and/or shapes. Features of openings 138 (e.g., a size and shape of pores of sponge 104) may be selected based on a location of treatment within the body, properties of a wound to be treated, a stage of treatment, or other factors. Sponge 104 may be any shape, including spherical, cylindrical, prismatic, cuboidal, irregular, or the like. Sponge 104 may be compressed into a lower profile during insertion to wound cavity 155 and may expand during deployment at wound cavity 155.

Sponge 104 may include any suitable biocompatible material that may absorb fluids and/or permit fluids or other materials to pass therethrough via, e.g., negative pressure applied to sponge 104. The material of sponge 104 may be flexible, compressible, porous, hydrophilic, sterile, and/or disposable. The material of sponge 104 may be an open-cell foam. Suitable materials include polyurethanes, esters, ethers, composite materials, and/or other medical-grade materials.

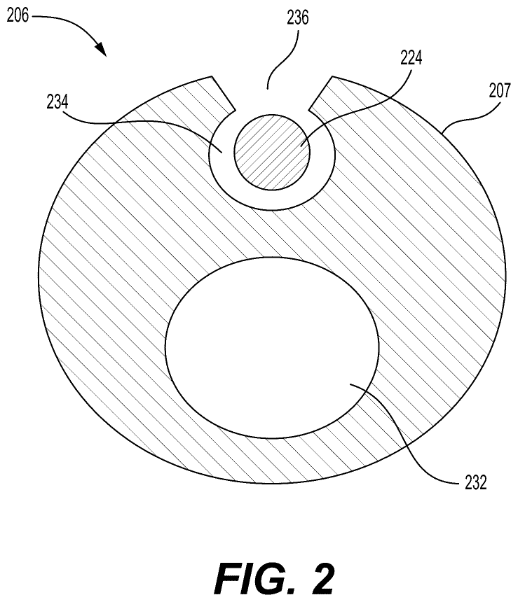

Sponge 104 may be coupled to a distal portion 108 of tube 106 via a coupling mechanism 105, e.g., sutures or other ties, an adhesive, a shrink-wrapped material, elastic(s), or the like. Distal portion 108 of tube 106 may extend longitudinally through sponge 104. In some examples, a recess may be provided in sponge 104 to receive distal portion 108 of tube 106. Tube 106 may include an outer wall 107 defining a lumen 110 in fluid communication with sponge 104. Lumen 110 may be open at both a proximal end and a distal end of tube 106. For example, lumen 110 may terminate at a distal opening 145 of tube 106. The proximal end of tube 106 may be connected to a vacuum or suction source, which may supply negative pressure to sponge 104. For example, a negative pressure of approximately 125 mm Hg, or approximately 2.5 pounds per square inch (PSI), may be supplied to sponge 104. Other suitable amounts of negative pressure may be used. The negative pressure may pull fluid and/or material from wound cavity 155 into lumen 110 of tube 106 via openings 138 of sponge 104 and distal opening 145 of tube 106 and/or pull tissue within wound cavity 155 towards sponge 104. In some examples, outer wall 107 of tube 106 may include a plurality of openings around a circumference of distal portion 108 of tube 106 and in fluid communication with lumen 110, which may increase the flow of fluid and/or other material into lumen 110 (see, e.g., FIG. 3).

As shown in FIG. 1A, medical device 102 may include an attachment device 116 (e.g., a T-tag or any other suitable type of tag, suture, rivet, anchor, or other coupler) coupled to sponge 104 and tube 106. Attachment device 116 may be disposed proximate a distalmost end 160 of sponge 104 and/or proximate distal opening 145 tube 106 (i.e., proximate the distal end of tube 106). In other words, attachment device 116 may be positioned distally of a proximal end of sponge 104 and a proximal portion of tube 106 that is within sponge 104. As discussed above, distal portion 108 of tube 106 may extend through sponge 104. A location of attachment device 116 may be longitudinally separated from a location of coupling mechanism 105 by a length of distal portion 108 within sponge 104. In examples, sponge 104 may have a length ranging from about 20 mm to about 100 mm. In these instances, a distance between distalmost end 160 of sponge 104 and attachment device 116 may range from about 10 mm to about 50 mm. In aspects, attachment device 116 may be located in a distal half of distal portion 108 of tube 016 that is within sponge 104.

In some aspects, attachment device 116 may include a stem portion 118 and a head portion 120. Attachment device 116 may have a substantially “T” shape, with stem portion 118 being the stem of the “T” and head portion 120 being the cross-bar of the “T.” Stem portion 118 may extend through a side of sponge 104 and/or wall 107 of tube 106 and terminate distally within lumen 110 of tube 106. Head portion 120 may be disposed along an exterior surface of wall 107 of tube 106. Stem portion 118 may extend within lumen 110 of tube 106 approximately perpendicular to a central longitudinal axis of tube 106. Stem portion 118 may be directly coupled to a thread 124 (e.g., a suture style thread, string, wire, etc.). Head portion 120 may be a flat or elongated portion that extends approximately perpendicular to stem portion 118 and may prevent separation of attachment device 116 from sponge 104 and tube 106, e.g., when thread 124 is pulled proximally. In some examples, one or both of stem portion 118 and head portion 120 may be rigid to maintain a position of attachment device 116 with respect to tube 106.

In some other examples, stem portion 118 may extend through sponge 104 approximately perpendicular to the central longitudinal axis of tube 106, and head portion 120 may be disposed within lumen 110 of tube 106. In other examples, stem portion 118 may extend through sponge 104 and tube 106 approximately perpendicular to the central longitudinal axis of tube 106, and head portion 120 may be disposed along an exterior of sponge 104 (e.g., along a side wall of sponge 104). Attachment device 116 may be any suitable size to allow negative pressure to be supplied to sponge 104 through tube 106. Although a t-tag structure is described above and may be advantageous due to manufacturing and structural simplicity, as well as anchoring effectiveness, it will be appreciated that attachment device 116 may have other suitable structures that are configured to couple thread 124 to sponge 104 and/or tube 106.

As mentioned above, thread 124 may be directly coupled to stem portion 118 of attachment device 116, which may facilitate removal of sponge 104 from wound cavity 155. Although the term “thread” is used herein, thread 124 (and other threads disclosed herein) may include any suitable type of string, suture, filament, wire, or cable, formed of any suitable manner. As shown in FIG. 1A, both ends of thread 124 may be coupled to stem portion 118 forming a loop 126. In other words, thread 124 may loop or fold back on itself at a proximal end. Loop 126 may extend alongside a proximal portion 114 of sponge 104. A proximalmost end of loop 126 may be proximal of a proximalmost end of sponge 104. In other words, loop 126 may extend proximally from attachment device 116. As shown in FIG. 1B, loop 126 may extend proximally out of wound cavity 155 and into body lumen 150 when sponge 104 is positioned within wound cavity 155. To remove sponge 104 from wound cavity 155, an operator may use a grasper or other medical device to grasp loop 126 and pull loop 126 proximally, which in turn may apply a pulling force on attachment device 116. Attachment device 116 may transfer the pulling force through tube 106 and sponge 104, which may help prevent tube 106 from separating from sponge 104 during removal of sponge 104 from wound cavity 155.

An arrangement of attachment device 116, sponge 104, tube 106, and loop 126 may be advantageous as compared to conventional systems by inhibiting separation of tube 106 from sponge 104. In conventional systems, a force on a tube or on a removal element may be in a longitudinal direction (e.g., coaxial with the tube). This longitudinal force may cause the tube to pull out of or otherwise separate from the sponge. In contrast, a force exerted on tube 106 by loop 126 may be at a non-zero angle relative to a longitudinal axis of tube 106. The force may cause tube 106 and/or sponge 104 to bend as sponge 104 is removed from wound cavity 155. Furthermore, because attachment device 116 extends at a non-zero angle (e.g., at an approximately 90 degree angle) relative to a central longitudinal axis of sponge 104 and tube 106 through both tube 106 and sponge 104, attachment device 116 may inhibit sponge 104 from becoming uncoupled from tube 106. A longitudinal separation between coupling mechanism 105 and attachment device 116 may result in the non-zero angle of the force on tube 106/sponge 104 and may otherwise inhibit tube 106 from separating from sponge 104.

Although medical device 102 is illustrated as having one attachment device 116 in FIG. 1A, it will be appreciated that medical 102 may include more than one attachment device 116. For example, medical device 102 may include a plurality of attachment devices 116 tied together to improve robustness. In other examples, medical device 102 may include a plurality of attachment devices 116 at various positions along sponge 104 and tube 106. Each attachment device 116 may be coupled to a single, separate thread 124 providing an operator with more than one option for removing sponge 104 from wound cavity 155. Each of the threads 124 may be a different color corresponding to a position of its corresponding attachment device 116.

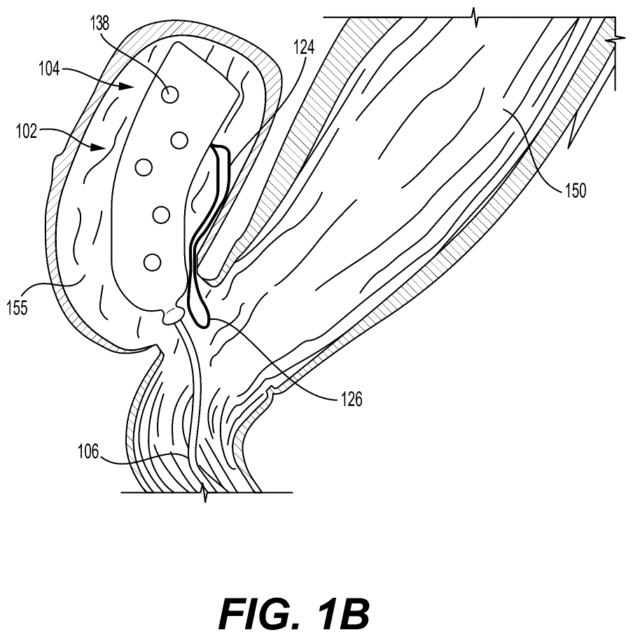

FIG. 2 illustrates a cross-sectional view of an exemplary tube 206 that may be used with sponge 104 and attachment device 116 (e.g., in place of tube 106). Tube 206 may have any of the features of tube 106, unless otherwise specified herein. In this example, tube 206 may include a first channel 232 configured to couple to a vacuum or suction source for supplying negative pressure to sponge 104, and a second channel 234 configured to receive thread 124. Tube 206 may further include a slit 236 extending through an outer wall 207 of tube 206, wherein slit 236 may be in communication with second channel 234. Thread 224 may extend proximally from attachment device 116 and within second channel 234. Alternatively, attachment device 116 may be omitted and thread 224 may be directly coupled to a distal end of tube 106 (e.g., within a distal portion of second channel 234 that does not have slit 236). Thread 224 may be movable from a position within second channel 234 to a position outside of tube 206 via slit 236.

Thread 224 may be disposed within second channel 234 until an operator is ready to remove sponge 104 from wound cavity 155. For example, slit 236 may extend to a portion of tube 206 that is proximal of a proximalmost end of sponge 104. In some examples, a proximal end of thread 224 may extend out of slit 236 so that the proximal end of thread 224 is outside of tube 206 and accessible to an operator. For example, thread 224 may have a greater length than slit 236, such that a proximalmost end of thread 124 is proximal of a proximalmost end of slit 236. Alternatively, thread 224 may extend laterally out of slit 236 and may be retained outside of tube 206 via, e.g., a thickened portion of thread 124.

To remove sponge 104, the operator may use a grasper or other medical device to grasp thread 224 (e.g., loop 126) and move more distal portions of thread 224 out of second channel 234 via slit 236. The operator may then pull thread 224 proximally to remove sponge 104 from wound cavity 155. The advantages of the arrangement of tube 206 and thread 224 may include any of the advantages discussed above with respect to FIGS. 1A and 1B. Thread 224 being disposed within second channel 234 prior to removal may help ensure that thread 224 is visible to the operator. That is, second channel 234 may prevent thread 224 from getting buried within sponge 104 and/or wound cavity 155.

FIG. 3 illustrates a distal portion of an exemplary medical device 302 including a sponge 304 and a tube 306. A distal portion 308 of tube 306 may be received within sponge 304. Sponge 304 may have any of the features of sponge 104, unless otherwise specified herein, and tube 306 may have any of the features of tubes 106, 206, unless otherwise specified herein. Sponge 304 may include a plurality of openings 338 in fluid communication with a lumen 310 of tube 306. Lumen 310 may be defined by an outer wall 307 of tube 306, and may be open at both proximal and distal ends of tube 306. For example, lumen 310 may terminate at a distal opening 345. The proximal end of tube 306 may be connected to a vacuum or suction source for applying negative pressure to sponge 304. Outer wall 307 may include a plurality of openings 342 around a circumference of distal portion 308 of tube 306 and in fluid communication with lumen 310.

Distal portion 308 of tube 306 may be coupled to a helical body 344 (e.g., a coiled body). In examples, helical body 344 may be integral with tube 306 (formed from a single, monolithic piece of material). Helical body 344 may extend distally from distal opening 345 of tube 306 (i.e., from the distal end of tube 306) to a tip 349 of helical body 344, while still allowing negative pressure to be applied to sponge 304, e.g., via openings 342, 345 of tube 306. For example, coils/turns of helical body 344 may define a central opening that is coaxial with opening 345. Helical body 344 may allow for distal portion 308 of tube 306 to be screwed into sponge 304. Helical body 344 may increase the mechanical connection between sponge 304 and tube 306, and may prevent tube 306 from separating from sponge 304, e.g., when tube 306 is pulled proximally.

Sponge 304 may be further secured to tube 306 via a thread 324 (e.g., a suture style thread, string, wire, etc.) wrapped around a proximal portion 314 of sponge 304 and distal portion 308 of tube 306. Optionally, sponge 304 may include a pre-formed cavity having a shape complementary to a shape of helical body 344, and helical body 344 may be screwed into the cavity. Alternatively, helical body 344 may be screwed into sponge 304 without such a cavity. In an alternative, helical body 344 may be omitted, and distal portion 308 of tube 306 may be helical or coiled (e.g., tube 306 may itself have a coiled shape, with lumen 310 extending through the coiled portion).

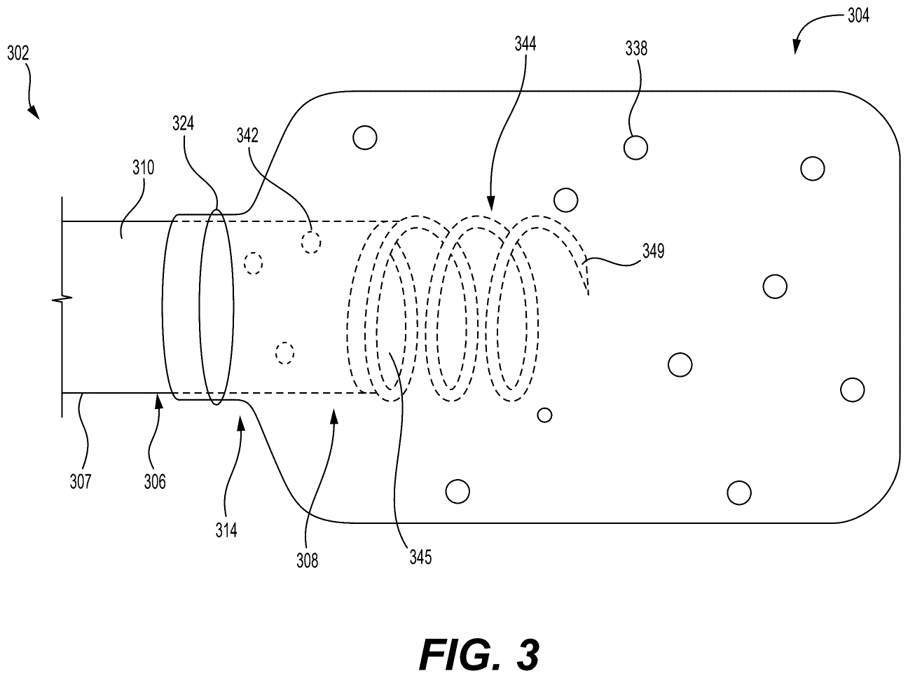

FIG. 4 illustrates a distal portion 408 of an exemplary tube 406 that may be used with sponges 104, 304 (e.g., in place of tubes 106, 306) or another sponge. Tube 406 may include any of the features of tubes 106, 306, unless otherwise specified herein. Tube 406 may include a lumen 410 defined by an outer wall 407 of tube 406. Outer wall 407 may optionally include a plurality of openings 442 around a circumference of distal portion 408 of tube 406 and in fluid communication with lumen 410. Lumen 410 may be open at both proximal and distal ends of tube 406, and the proximal end of tube 406 may be connected to a vacuum or suction source for applying negative pressure. In this example, distal portion 408 of tube 406 may include thread(s) 444 (e.g., a helical body, male screw threads) extending around outer wall 407 of tube 406. Threads 444 may allow distal portion 408 of tube 406 to be screwed into sponges 104, 304 or another sponge. Threads 444 may increase the mechanical connection between distal portion 408 of tube 406 and sponges 104, 304 or another sponge, and may prevent tube 406 from separating from sponges 104, 304 or another sponge, e.g., when tube 406 is pulled proximally. In examples, a thread pitch of threads 444 may range from about 1 mm to about 20 mm, although other distances are contemplated.

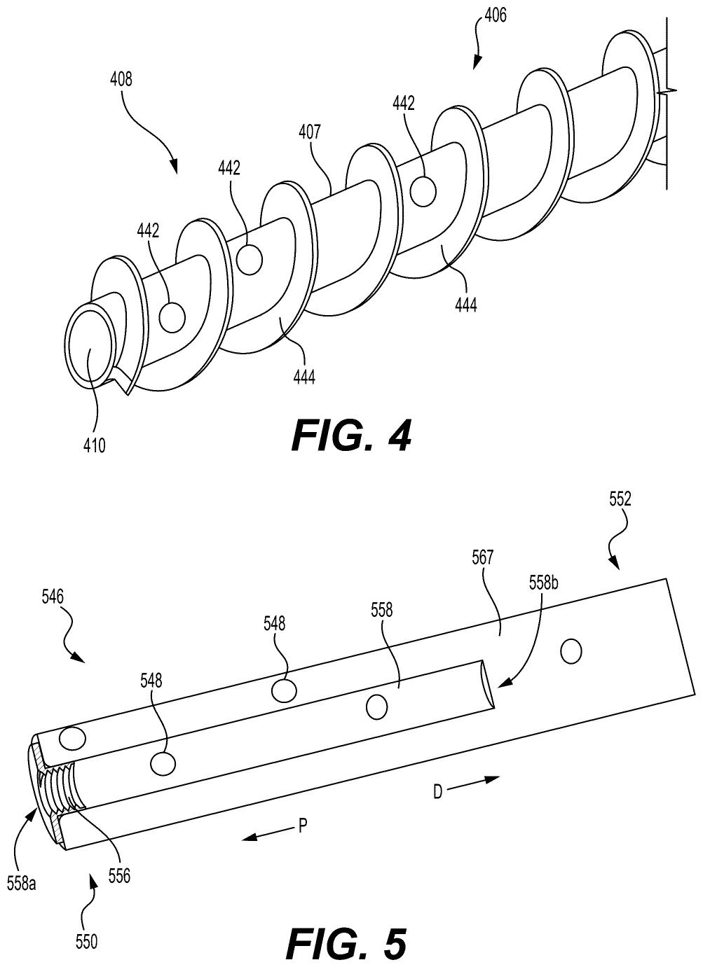

Tube 406 may be used in conjunction with a coupler (such as that discussed below with respect to FIG. 5) or directly with a sponge (e.g., sponge 104, 304). For example, a sponge (e.g., sponge 104, 304) may include a bore, which may or may not have female threads formed therein. Alternatively, tube 406 may be screwed into a sponge (e.g. sponge 104, 304) that does not have a pre-formed bore.

FIG. 5 illustrates an exemplary coupler 546 (e.g., an insert) configured to be received within sponges 104, 304 or another sponge. For example, coupler 546 may be fixed to sponges 104, 304 or another sponge, e.g., via any suitable adhesive (e.g., epoxy), by a friction fit, or by other mechanical fasteners. Coupler 546 may be configured to couple a tube having threads (e.g., female or male screw threads) to sponges 104, 304. For ease of discussion, coupler 546 will now be described in combination with tube 406.

Coupler 546 may include a body 567 having a lumen 558 sized and shaped to receive distal portion 408 of tube 406. Lumen 558 may include a proximal opening 558a and a distal opening 558b. Alternatively, distal opening 558b may be omitted (e.g., where tube 406 has openings 442). As shown in FIG. 5, lumen 558 may extend distally from a proximal end 550 of body 567 and may terminate proximally of a distal end 552 of body 567. Lumen 558 may include threads 556 (e.g., female screw threads) corresponding to threads 444 of distal portion 408 of tube 406 to allow for attachment of tube 406 to coupler 546. For example, distal portion 408 of tube 406 may be received within proximal opening 558a and screwed into lumen 558 to secure tube 406 to coupler 546. Threads 556 may extend along a partial length (e.g., a proximal or distal portion) or an entire length of lumen 558.

Body 567 may be porous to allow fluid communication between lumen 410 of tube 406 and sponges 104, 304 or another sponge. For example, coupler 546 may include a plurality of openings 548 dispersed throughout body 567, which may allow negative pressure to be supplied to sponges 104, 304 when distal portion 408 of tube 406 is secured within lumen 558. Alternatively, lumen 558 may extend along an entire length of body 567, such that distal opening 558b of lumen 558 may be at a distalmost end of body 567. In this alternative, openings 548 may be omitted from body 567 and distal opening 558b may open directly into sponges 104, 304.

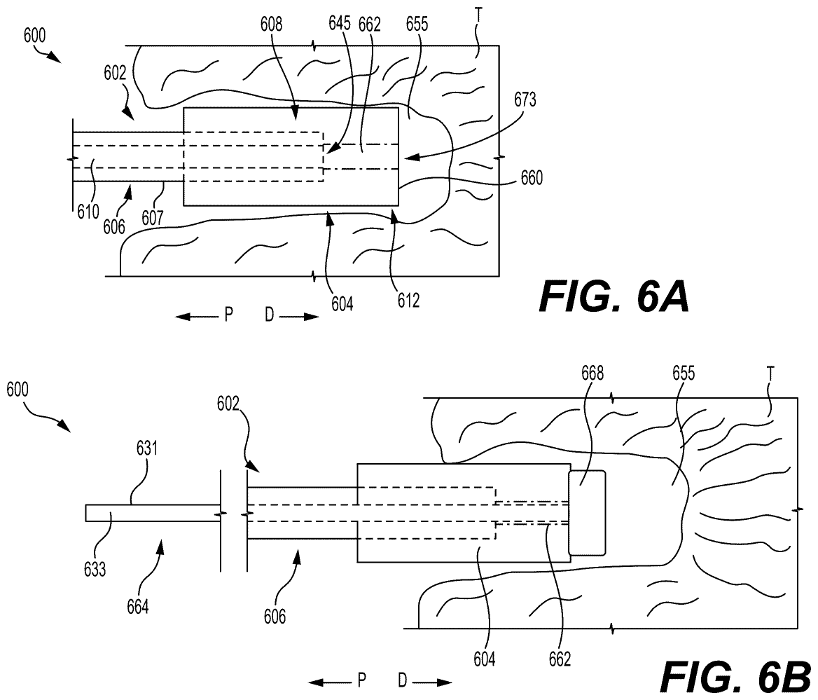

FIGS. 6A and 6B illustrate an exemplary medical system 600 within a body lumen (e.g., the esophagus) for treating a wound cavity 655 (e.g., a leak, an esophageal fistula, etc.). Medical system 600 may include a medical device 602 having a sponge 604 coupled to a distal portion 608 of a tube 606. Sponge 604 may have any of the features of sponges 104, 304, unless otherwise specified herein, and tube 606 may have any of the features of tubes 106, 206, 306, 406, unless otherwise specified herein. Tube 606 may include an outer wall 607 defining a lumen 610. Lumen 610 may be open at both proximal and distal ends of tube 606. For example, lumen 610 may terminate at a distal opening 645. The proximal end of tube 606 may be connected to a vacuum or suction source, which may supply a negative pressure to sponge 604. Outer wall 607 may include a plurality of openings around a circumference of distal portion 608 of tube 606 and in fluid communication with lumen 610.

Tube 606 may terminate proximally of a distalmost end 660 of sponge 604. Distal portion 608 of tube 606 may be oriented within sponge 604 such that lumen 610 of tube 606 may be aligned with and/or in fluid communication with a lumen 662 at a distal portion 612 of sponge 604. Lumen 662 of sponge 604 may extend distally from a position adjacent to distal opening 645 to an opening 673 at distalmost end 660 of sponge 604. In some examples, lumen 662 may extend through an entire length of sponge 604, and tube 606 may be positioned within a proximal portion of lumen 662. Alternatively, lumen 662 may only extend through a portion of sponge 604 that is distal to tube 606. Lumen 662 of sponge 604 may be an extension of lumen 610 of tube 606, e.g., for applying negative pressure and/or extending other medical devices through opening 673.

As shown in FIG. 6B, medical system 600 may further include a removal device 664 configured to assist with removal of sponge 604 from wound cavity 655. Removal device 664 may include an elongated member 631 (e.g., a shaft) defining a lumen 633. Lumen 633 may be open at both proximal and distal ends of elongated member 631. Alternatively, lumen 633 may have a closed distal end, and elongated member 631 may include openings formed in sidewall(s) of a distal portion of elongated member 631 that are in fluid communication with an area outside of elongated member 631. The distal end of elongated member 631 may be coupled to an inflatable balloon 668 or other expandable element, and the proximal end of elongated member 631 may be coupled to a source of fluid (e.g., saline or air) to inflate (e.g., expand) balloon 668. Balloon 668 may configured to transition between a deflated (e.g., collapsed) configuration to an inflated (e.g., expanded) configuration. Although balloon 668 is illustrated as having an approximately disc or donut in the inflated configuration in FIG. 6B, it will be appreciated that balloon 668 may have any suitable shape when inflated, e.g., a mushroom cap shape, a rectangular shape, a spherical or orb shape, a cylindrical shape etc.

During an exemplary medical procedure, an operator may position sponge 604 within wound cavity 655, as shown in FIG. 6A. The operator may then activate a vacuum source to apply negative pressure to sponge 604, which may pull fluid and/or material from wound cavity 655 into lumen 610 of tube 606 and/or pull tissue T within wound cavity 655. As wound cavity 655 heals, wound cavity 655 may shrink and become smaller, as shown in FIG. 6B. The size of sponge 604 may no longer be suitable for the adjusted size of wound cavity 655, and may need to be replaced with a smaller sponge 604 or otherwise removed.

To remove sponge 604 from wound cavity 655, the operator may disconnect the vacuum source from tube 606, and may advance balloon 668 in the deflated configuration through lumens 610, 662 and out of opening 673. With balloon 668 positioned distal of distalmost end 660 of sponge 604, the operator may then activate a fluid source at the proximal end of elongated member 631 to introduce fluid into lumen 633 to inflate balloon 668. As balloon 668 is inflated, balloon 668 may separate sponge 604 from tissue T (e.g., granulated tissue) within wound cavity 655. The operator may then pull longitudinal member 631 proximally to pull balloon 668 proximally against distalmost end 660 of sponge 604, and thusly remove sponge 604 from wound cavity 655. Removal device 664 may prevent sponge 604 from separating from tube 606 during removal of sponge 604 from wound cavity 655 and/or may facilitate easy peeling of sponge 604 from tissue T within wound cavity 655. For example, balloon 668 may exert a proximal force on distalmost end 660 of sponge 604 to move both sponge 604 and tube 606 proximally, rather than an operator pulling on tube 606 itself, separately from sponge 604.

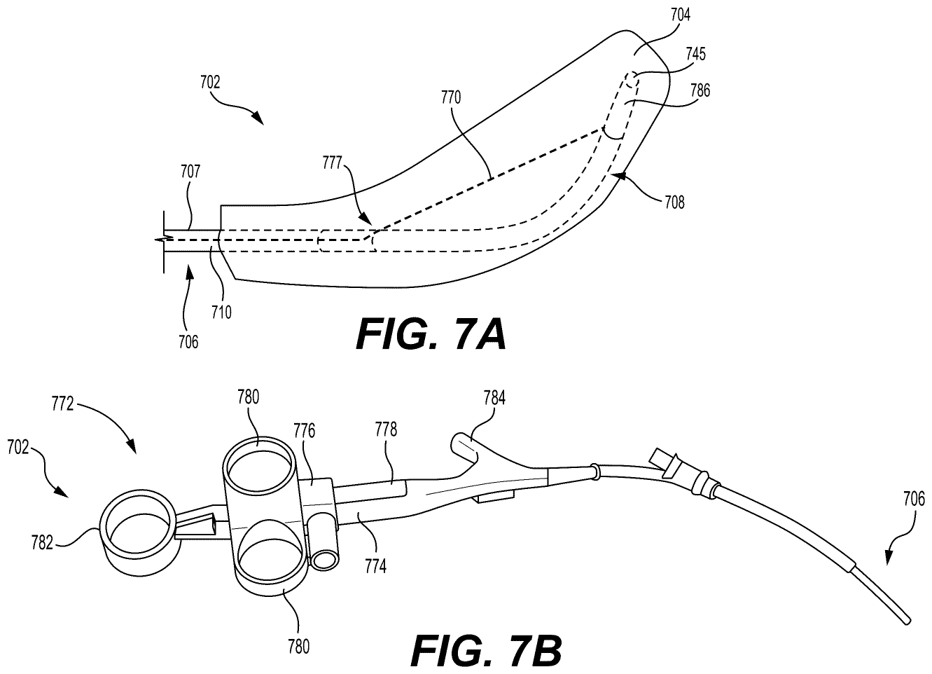

FIGS. 7A and 7B illustrate features of an exemplary medical device 702 including a sponge 704 and a tube 706. FIG. 7A illustrates a distal portion 708 of tube 706 within sponge 704 and in an actuated configuration (e.g., a bent configuration). FIG. 7B illustrates an exemplary handle 772 for use with sponge 704 and tube 706. Handle 772 may alternatively be used with other aspects disclosed herein, such as removal device 664. Sponge 704 may have any of the features of sponges 104, 304, 604, unless otherwise specified herein, and tube 706 may have any of the features of tubes 106, 206, 306, 406, 606, unless otherwise specified herein.

Tube 706 may extend from a proximal end coupled to handle 772 to a distal tip 786. Tube 706 may include an outer wall 707 defining a lumen 710 and lumen 710 may terminate at a distal opening 745 of tube 706. Lumen 710 may be in communication with a port 784 of handle 772 and port 784 may be configured to couple to a vacuum or suction source for applying negative pressure to sponge 704 via lumen 710.

Medical device 702 may include a control wire 770 extending from a proximal end to a distal end. The distal end of control wire 770 may be fixed to or near distal tip 786 and may extend proximally to handle 772 or other actuator for actuating (e.g., bending) distal portion 708 of tube 706. Distal portion 708 of tube 706 may be configured to transition between actuated (e.g., bent) and unactuated (e.g., linear) configurations. Movement of distal portion 708 of tube 706 may correspondingly move sponge 704. As shown in FIG. 7A, control wire 770 may extend proximally from distal tip 786 and outside of tube 706, and may enter lumen 710 via a slot 777 of tube 706. Control wire 770 may be formed of nitinol or other metal, which may allow the portion of control wire 770 extending outside of tube 706 to cut through portions of sponge 704 when pulled proximally and minimize a level at which sponge 704 may interfere within bending of distal portion 708 of tube 706.

Referring to FIG. 7B, handle 772 may include a main body 774 and a movable body 776 (e.g., an actuator) movable relative to main body 774. Main body 774 may include a slot 778 and a proximal ring 782 (e.g., to receive an operator's thumb). Movable body 776 may be slidably positioned within slot 778 of main body 774 and may include rings 780 (e.g., to receive an operator's index and middle fingers). The proximal end of control wire 770 may be fixed to a movable body 776, such that movement of movable body 776 along slot 778 may control bending of distal portion 708 of tube 706. For example, proximal movement of movable body 776 along slot 778 may pull control wire 770 proximally and transition distal portion 708 of tube 706 to the bent configuration, as shown in FIG. 7A. For example, distal movement of movable body 776 along slot 778 may move control wire 770 distally and transition distal portion 708 of tube 706 to the linear configuration.

During an exemplary medical procedure, sponge 704 and distal portion 708 of tube 706 may be delivered and positioned within a wound cavity (e.g., a leak, an esophageal fistula, etc.) in the linear (unbent) configuration. Negative pressure may then be applied to sponge 704, which may pull fluid and/or material from the wound cavity into lumen 710 of tube 706 via opening 745 and/or pull tissue within the wound cavity inwards towards sponge 704. After a suitable period of time, the operator may remove sponge 704 from the wound cavity. To remove sponge 704 from the wound cavity, the operator may move movable body 776 proximally along slot 778 of main body 774 to pull control wire 770 proximally and bend distal portion 708 of tube 706 and sponge 704. The operator may move movable body 776 proximally and distally along slot 778 of main body 774 to transition distal portion 708 of tube 706 between the bent and linear configurations. Back and forth movement of distal portion 708 of tube 706 between the bent and linear configurations may help peel sponge 704 away or otherwise detach sponge 704 from tissue within the wound cavity. The operator may then pull tube 706 proximally to remove sponge 704 from the wound cavity.

As discussed above, in conventional systems, a force on a tube or on a removal element may be in a longitudinal direction (e.g., coaxial with the tube). This longitudinal force may cause the tube to pull out of or otherwise separate from the sponge. In this example, a force exerted on tube 706 by control wire 770 may be at a non-zero angle relative to a longitudinal axis of tube 706, which may cause distal portion 708 of tube 706 and sponge 704 to bend. Removing sponge 704 from the wound cavity while distal portion 708 of tube 706 is in the bent configuration may prevent tube 706 from separating from sponge 704 when tube 706 is pulled proximally because a force exerted on tube 706 may be at a non-zero angle relative to the longitudinal axis of tube 706.

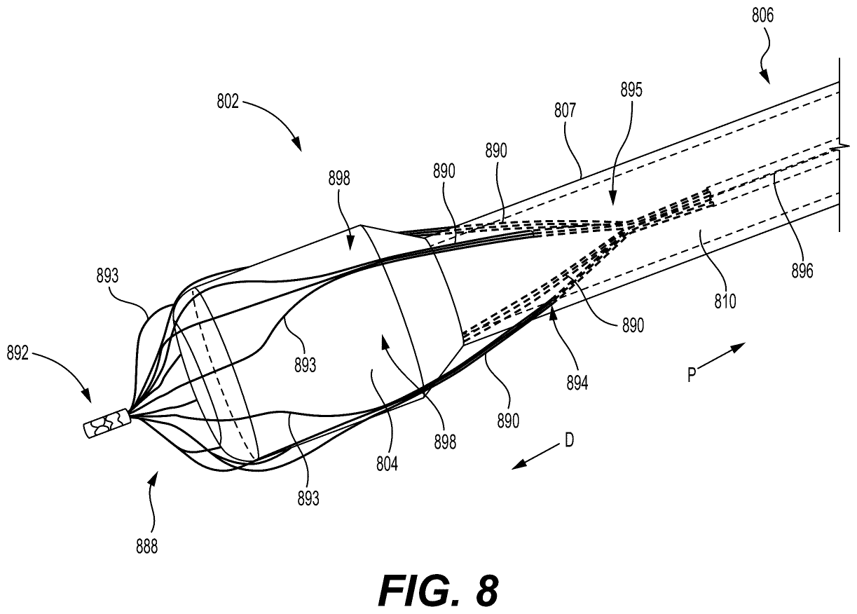

FIG. 8 illustrates a distal portion of another exemplary medical device 802. Medical device 802 may include a sponge 804 coupled to a distal portion of a tube 806. Sponge 804 may have any of the features of sponges 104, 304, 604, 704, unless otherwise specified herein, and tube 806 may have any of the features of tubes 106, 206, 306, 406, 606, 706, unless otherwise specified herein. Tube 806 may include an outer wall 807 defining a lumen 810 configured to supply a negative pressure to sponge 804.

Medical device 802 may further comprise a basket 888 enclosing sponge 804. Basket 888 may have any of the features of any medical basket known in the art. Basket 888 may include a plurality of legs 890 and a plurality of strands 893. For example, legs 890 and strands 893 may be wires. Basket 888 may include four legs 890 coupled to one another and/or joined together at a proximal end 895 of basket 888. Each of the four legs 890 may extend distally from within lumen 810 and may exit out of tubes 806 via slots 894 of tube 806. Legs 890 may be movable through slots 894. Alternatively, an overtube may be positioned over tube 806, and the legs may extend out of a distal end of the overtube.

Each of the four legs 890 may diverge into strands 893 and strands 893 may converge at a distal end 892 of basket 888. Although basket 888 is shown with a distal tip at distal end 892, it will be appreciated that basket 888 may lack such a distal tip to decrease trauma to body tissues. Legs 890 and strands 893 may form a plurality of apertures 898 which may be sized to permit fluid communication between sponge 804 and a wound cavity. Apertures 898 may also allow an operator to cut sponge 804 through basket 888 to a desired size, e.g., a size corresponding to a wound cavity. It will be appreciated that basket 888 may include any number or pattern of legs 890 and strands 893.

Legs 890 of basket 888 may be directly coupled to one or more control wire(s) 896 extending within lumen 810 for actuating basket 888. Alternatively, control wire 896 may extend between tube 806 and an overtube positioned over tube 806. Proximal movement of control wire(s) 896 may close (e.g., collapse) basket 888 around sponge 804 and may compress sponge 804. Distal movement of control wire(s) 896 may open (e.g., expand) basket 888 and may allow sponge 804 to expand. Control wire(s) 896 may extend through lumen 810 without interfering with the application of negative pressure through lumen 810. Alternatively, tube 806 may include two separate lumens, e.g., a first lumen and a second lumen. In this alternative, control wire(s) 896 may extend through the first lumen and negative pressure may be applied through the second lumen.

Control wire(s) 896 may extend from proximal end 895 of basket 888 and to an actuator at a proximal end of medical device 802. In some aspects, sponge 804, tube 806, and basket 888 may be used in combination with handle 772 of FIG. 7B. For example, the proximal end of tube 806 may be coupled to handle 772 and control wire(s) 896 may be coupled to movable body 776 for controlling proximal and distal movement of control wire(s) 896. Lumen 810 may be in fluid communication with port 784 of handle 772 and port 784 of handle 772 may be coupled to a vacuum or suction source for supplying negative pressure to sponge 804 via lumen 810.

During an exemplary medical procedure, an operator may pull control wire(s) 896 proximally to close basket 888 around sponge 804 and compress sponge 804. Sponge 804 may optionally be delivered and positioned within a wound cavity in a compressed state. The operator may then move control wire(s) 896 distally to open basket 888 and expand sponge 804. Negative pressure may then be applied to sponge 804, which may pull fluid and/or material from the wound cavity into lumen 810 of tube 806 and/or pull tissue within the wound cavity inwards towards sponge 804. After a suitable period of time, the operator may remove sponge 804 from the wound cavity. To remove sponge 804 from the wound cavity, the operator may move control wire(s) 896 proximally to collapse basket 888 around sponge 804. Closing of basket 888 around sponge 804 may help peel or otherwise detach sponge 804 from tissue within the wound cavity. Basket 888 may also help prevent separation of sponge 804 from tube 806 during removal and delivery of sponge 804 to a wound cavity by holding or capturing sponge 804. A smaller, compressed size of sponge 804 may also facilitate removing sponge 804 through a body lumen.

In an alternative, basket 888 may include only two legs 890 joined together at proximal end 895 and distal end 892, which may provide for larger sized apertures 898 and/or may reduce a compressive force on sponge 804, e.g., when basket 888 is in the closed configuration. For example, basket 888 may have a snare shape. Basket 888 including only two legs 890 may allow for an operator to more easily cut sponge 804 around basket 888. Additionally, basket 888 including only two legs 890 may help encourage more granulation of tissue and retention of fluids and/or material within sponge 804 during removal of sponge 804.

It will be apparent to those skilled in the art that various modifications and variations may be made in the disclosed devices and methods without departing from the scope of the disclosure. Other aspects of the disclosure will be apparent to those skilled in the art from consideration of the specification and practice of the features disclosed herein. It is intended that the specification and examples be considered as exemplary only.

We claim:

1. A medical device comprising:
a tube defining a lumen, wherein the tube is configured to couple to a source of suction;
a porous body coupled to a distal portion of the tube, wherein the porous body includes a plurality of openings in fluid communication with the lumen; and
a wire coupled to the distal portion of the tube, wherein the wire is configured to move the distal portion of the tube and the porous body via a pulling force applied to the wire, and wherein the pulling force applied to the wire exerts a force on the tube that is at a non-zero angle relative to a central longitudinal axis of the tube.

2. The medical device of claim 1, further comprising an attachment device extending through at least a portion of the porous body and the tube, wherein the attachment device is coupled to the wire.

3. The medical device of claim 2, wherein the attachment device is proximate a distalmost end of the porous body.

4. The medical device of claim 2, wherein the attachment device includes a stem portion and a head portion, wherein the stem portion extends through the lumen of the tube approximately perpendicularly the central longitudinal axis of the tube, and wherein the head portion is disposed exterior of the tube and extends perpendicular to the stem portion.

5. The medical device of claim 2, wherein ends of the wire are coupled to the attachment device forming a loop.

6. The medical device of claim 5, wherein the loop extends proximally from the attachment device when the porous body is within a body lumen.

7. The medical device of claim 1, wherein the lumen of the tube is a first lumen of the tube, and wherein the tube further defines a second lumen.

8. The medical device of claim 7, wherein the wire is received within the second lumen.

9. The medical device of claim 7, wherein the tube includes a slit extending through a wall of the tube and in fluid communication with the second lumen, and wherein the wire is movable from a position within the second lumen to a position outside of the tube via the slit.

10. The medical device of claim 1, wherein the wire is configured to transition the distal portion of the tube between a linear configuration and a bent configuration.

11. The medical device of claim 10, further comprising a handle coupled to a proximal end of the tube and including an actuator, wherein the wire extends proximally from the distal portion of the tube to the actuator.

12. The medical device of claim 11, wherein the actuator is configured to move the wire in a proximal direction or a distal direction.

13. The medical device of claim 11, wherein the handle includes a port in fluid communication with the lumen of the tube and configured to couple to the source of suction.

14. The medical device of claim 1, wherein a portion of the wire extends outside of the tube and within the porous body.

15. The medical device of claim 14, wherein the portion of the wire is configured to cut the porous body.

16. A medical device comprising:
a porous body including a plurality of openings;
a tube defining a lumen in fluid communication with the plurality of openings of the porous body, wherein a proximal end of the tube is configured to couple to a source of suction and a distal end of the tube is coupled to the porous body; and
a helical body coupled to the distal end of the tube.

17. The medical device of claim 16, wherein the helical body extends distally from a distal opening of the lumen of the tube.

18. The medical device of claim 16, wherein the helical body extends around the distal end of the tube.

19. A medical system comprising:
a tube extending from a proximal end to a distal end and defining a lumen, wherein the proximal end of the tube is configured to couple to a source of suction;
a porous body coupled to a distal end of the tube, wherein the porous body includes a lumen at a distal portion of the porous body in fluid communication with the lumen of the tube, wherein the lumen of the porous body includes an opening at a distalmost end of the porous body; and
a removal device including:
a shaft extending from a proximal end to a distal end and defining a lumen, wherein the proximal end of the shaft is configured to couple to a source of fluid; and
an inflatable balloon coupled to the distal end of the shaft and in fluid communication the lumen of the shaft.

20. The medical system of claim 19, wherein the inflatable balloon is configured to extend through the lumen of the tube and the lumen of the porous body.
